(19) United States
(12) Patent Application Publication
(10) Pub. No.: US 2008/0096885 A1
(43) Pub. Date: Apr. 24, 2008

(54) Quinoline Derivatives as Neurokinin Receptor Antagonists

(76) Inventors: William Robert Carling, Bishops Stortford (GB); Jason Matthew Elliott, Felsted (GB); Elena Mezzogori, Ravenna (IT); Michael Geoffrey Neil Russell, Welwyn Garden City (GB); Briam John Williams, Great Dunmow (GB)

Correspondence Address: MERCK AND CO., INC, P O BOX 2000, RAHWAY, NJ, 07065-0907, US

(21) Appl. No.: 11/659,399

(22) PCT Filed: Jul. 28, 2005
(86) PCT No.: PCT/GB05/50120

§ 371 Date: Aug. 13, 2007

(30) Foreign Application Priority Data
Aug. 6, 2004 (GB) 0417560.0

(51) Int. Cl. A61K 31/47; A61P 25/18; A61K 31/5377; C07D 215/20; C07D 413/02

(52) U.S. Cl. 514/235.2; 546/156; 544/128; 514/312

(57) ABSTRACT

The present invention relates to substituted quinoline-4-carboxylic acid hydrazides defined herein, pharmaceutical compositions comprising them and their use in treating diseases mediated by neurokinin-2 and/or neurokinin-3 (NK-3) receptors.

The present invention relates to substituted quinoline-4-carboxylic acid hydrazides defined herein, pharmaceutical compositions comprising them and their use in treating diseases mediated by neurokinin-2 and/or neurokinin-3 (NK-3) receptors. These compounds can thus be used in methods of treatment to suppress and treat such disorders.

Background information on NK-3 receptor antagonists can be found in literature reviews such as Giardina and Raveglia, Exp. Opin. Ther. Patents (1997) 7(4): 307-323 and Giardina et al, Exp. Opin. Ther. Patents (2000) 10(6): 939-960. These references also contain pertinent information on preclinical validation of therapies that can be treated with NK-3 antagonists.

Representative examples of compounds prepared in the art as NK-3 antagonists are to be found in WO-A-9719926 (SmithKline Beecham S.p.a.) and U.S. Pat. No. 5,741,910 (Sanofi). Structurally related compounds as NK-3 and/or NK-2 receptor antagonists are disclosed in International patent application no. PCT/GB2004/000415.

The present invention thus provides a compound of formula (I): 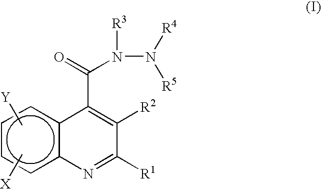
wherein:

R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulphur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;

or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄ alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a},

where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen,

or, when R¹ is CONR^{a}R^{b} or SO₂NR^{a}R^{b}, R^{a}, R^{b} and the nitrogen atom to which they are attached form a piperidine, piperazine, pyrrolidine, morpholine, aziridine, azetidine or azepine ring, optionally substituted by hydroxy, C₁₋₄alkyl or C₁₋₄alkoxy;

R² is hydrogen, hydroxy, halogen or CN;

or R² is C₁₋₆alkyl, C₂₋₆alkenyl, C₂₋₆alkynyl, C₃₋₈cycloalkyl, C₁₋₆alkoxy, (CH₂)₀₋₆NR^{c}R^{d}, C₁₋₆alkoxy substituted by NR^{c}R^{d}, OC₃₋₈cycloalkyl, OHet, Het, Oheteroaryl, heteroaryl, Oaryl, aryl, (CH₂)₀₋₄NR^{e}C(O)R^{f}, (CH₂)₀₋₄NR^{e}C(O)OR^{f}, (CH₂)₀₋₄NR^{e}S(O)₂R^{f}, SO₂R^{c}, SO₂NR^{c}R^{d}, COOR^{c}, C(O)R^{c}, C(O)NR^{c}R^{d},

optionally substituted by 1 to 8 halogen atoms,

where R^{c}, R^{d}, R^{e} and R^{f} are independently selected from hydrogen, C₁₋₆alkyl, C₂₋₆alkenyl, C₂₋₆alkynyl, C₃₋₈cycloalkyl and aryl,

or where R^{c}and R^{d}, together with the nitrogen atom to which they are attached, form a saturated nitrogen-containing 3-7 membered heterocycle optionally containing a further nitrogen or oxygen atom and optionally substituted by NR′R″,

where R′ and R″ are independently chosen from hydrogen and C₁₋₆alkyl,

or R^{e} and R^{f} are linked as a C₂₋₆alkylene, C₂₋₆alkenylene or C₃₋₆alkynylene group, optionally substituted by hydroxyl or halogen,

where Het is as hereinbelow defined;

R³ is hydrogen or C₁₋₆alkyl;

R⁴ is hydrogen, C₁₋₈alkyl, C₂₋₈alkenyl, C₂₋₈alkynyl, C₃₋₈cycloalkyl, aryl or arylC₁₋₆alkyl, optionally substituted by hydroxy, C₁₋₆alkoxy, CN, NH₂ or 1 to 8 halogen atoms;

or R⁴ is a moiety containing at least one aromatic ring and possessing 5, 6, 9 or 10 ring atoms of which 1, 2, 3 or 4 atoms are heteroatoms independently selected from N, O and S, which ring system is optionally substituted at any substitutable position by 1, 2 or 3 groups chosen from hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, which group is optionally substituted by 1 to 8 halogen atoms;

R⁵ is hydrogen, C₁₋₈alkyl, C₂₋₈alkenyl, C₂₋₈alkynyl, C₃₋₈cycloalkyl, aryl, arylC₁₋₆ alkyl, (CH₂)₀₋₄heteroaryl, (CH₂)₀₋₄Het, C(O)NR^{g}R^{h}, S(O)₂NR^{g}R^{h}, S(O)₂R^{g}, C(O)OR^{g} or C(O)R^{g},

where R^{g} and R^{h} are each independently selected from hydrogen, C₁₋₆alkyl, C₂₋₆alkenyl, C₂₋₆alkynyl, (CH₂)₀₋₄C₃₋₈cycloalkyl, (CH₂)₀₋₄aryl, (CH₂)₀₋₄heteroaryl and (CH₂)₀₋₄Het, optionally substituted by hydroxy and 1 to 8 halogen atoms;

or R⁴ and R⁵, together with the nitrogen atom to which they are attached, form a mono- or bicyclic moiety possessing 3 to 10 ring atoms of which optionally 1, 2, 3 or 4 atoms are heteroatoms independently selected from N, O and S, which ring system is optionally substituted at any substitutable position by 1, 2 or 3 groups chosen from halogen, NO₂, CN, NH₂, oxo, C₁₋₄alkyl, C₂₋₄alkenyl, C₂₋₄alkynyl and C₁₋₆alkoxy, which group is optionally substituted by 1 to 8 halogen atoms;

X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NRⁱR^{j}, CO₂Rⁱ, (CH₂)₀₋₄NR^{k}R^{m}, SO₂Rⁱ and SO₂NRⁱR^{j}, optionally substituted by halogen;

Rⁱ and R^{j} are independently chosen from hydrogen, C₁₋₆alkyl, C₂₋₆alkenyl, C₂₋₆alkynyl, C₃₋₈cycloalkyl and (CH₂)₀₋₄aryl;

R^{k} and R^{m} are independently chosen from hydrogen, C₁₋₆alkyl, C₂₋₆alkenyl, C₂₋₆alkynyl, C₃₋₈cycloalkyl, aryl, C(O)R^{p}, COOR^{p} and S(O)₂R^{p};

R^{p} is hydrogen, C₁₋₆alkyl, C₂₋₆alkenyl, C₂₋₆alkynyl, C₃₋₈cycloalkyl or (CH₂)₀₋₄aryl;

or a pharmaceutically acceptable salt thereof;
with the proviso that the compound of formula (I) is not: 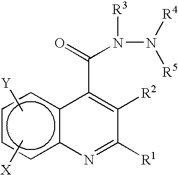
wherein:

R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulphur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;

R² is hydroxy, C₁₋₆alkoxy, C₁₋₆alkyl, amino, NR′R″ or C₁₋₆alkyl-NR′R″ where R′ and R″ are independently chosen from hydrogen and C₁₋₄alkyl and where R′ and R″, together with the nitrogen atom to which they are attached, form a saturated nitrogen-containing 3-7 membered heterocycle optionally containing a further nitrogen atom and optionally substituted by NR′R″ as defined above or R² is C₁₋₆alkoxy substituted by NR′R″ as defined above;

R³ is hydrogen or C₁₋₆alkyl;

R⁴ is hydrogen, C₁₋₈alkyl, C₂₋₈alkenyl, C₂₋₈alkynyl, C₃₋₈cycloalkyl, aryl or arylC₁₋₆alkyl;

R⁵ is hydrogen, C₁₋₈alkyl, C₂₋₈alkenyl, C₂₋₈alkynyl, C₃₋₈cycloalkyl, aryl, arylC₁₋₆alkyl or C₁₋₆alkoxycarbonyl;

or R⁴ and R⁵, together with the nitrogen atom to which they are attached, form a C₃-C₁₀ mono- or bicyclic saturated ring;

X and Y are independently chosen from hydrogen, hydroxy, nitro, amino, cyano, CF₃, halogen and C₁₋₄alkyl;

or a pharmaceutically acceptable salt thereof.

Examples of suitable R¹ groups include azabicyclo[3.2.2]nonanyl, dihydroquinolinyl, tetrahydroquinolinyl, dihydroisoquinolinyl and tetrahydroisoquinolinyl.

R¹ is preferably unsubstituted or monosubstituted by hydroxy, halogen, nitro, cyano, amino, CF₃ or CH₃. Preferably, R¹ is aryl, such as phenyl, or Het. More preferably, R¹ is 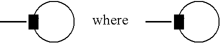
is defined as a 4- to 7-membered heteroaliphatic ring, containing 1, 2 or 3 heteroatoms selected from N, O or S or a group S(O), S(O)₂, NH or NC₁₋₄alkyl, which ring is attached through the nitrogen atom. Most preferably, R¹ is aziridine, azetidinyl, pyrrolidinyl, piperidinyl, azepinyl or piperazinyl, especially pyrrolidinyl or piperidinyl, where said ring is optionally substituted by 1, 2 or 3 groups independently chosen from hydroxy, halogen, NO₂, CN, NH₂, CF₃, C₁₋₄alkyl, C₂₋₄alkenyl and C₂₋₄alkynyl, preferably hydroxy, halogen, CF₃ or C₁₋₄alkyl, more preferably CF₃ or C₁₋₄alkyl.

Preferably, R² is C₁₋₆alkyl, C₁₋₆alkoxy or NR^{c}R^{d}, substituted by 1 to 8 halogen atoms, preferably 1 to 5 fluorine, chlorine or bromine atoms. Examples of suitable R² groups include CH₂F, CHF₂, CF₃, CH₂CF₃, OCF₃, OCH₂CF₃, N(H)CF₃, N(H)CH₂CF₃, N(CH₃)CF₃, N(CH₃)CH₂CF₃, N(CF₃)₂, N(CF₃)CH₂CF₃, N(CH₂CF₃)₂, N(CH₂CH₃)CH₂CF₃. Preferred R² groups are N(H)CH₂CF₃ and OCH₂CF₃.

Examples of suitable R⁴ moieties with 9 ring atoms include pyrazolopyrimidinyl, imidazopyridinyl, purinyl, indolyl, indolinyl, isoindolinyl, benzofuranyl, dihydrobenzofuranyl, phthalanyl, benzothienyl, indazolyl, benzimidazolyl, benzoxazolyl, benzisoxazolyl, benzothiazolyl, benzotriazolyl.

Examples of suitable R⁴ moieties with 10 ring atoms include phthalazinyl, quinazolinyl, quinoxalinyl, pyrimidopyrimidinyl, naphthyridinyl, pteridinyl, benzopyranyl, chromanyl, benzothiazinyl, quinolyl, isoquinolyl, pyridopyridinyl, pteridinyl.

Preferably, R⁴ is phenyl or a five- or six-membered aromatic ring containing 1, 2 or 3 heteroatoms selected from N, O and S, where R⁴ is optionally substituted at any substitutable position by 1, 2 or 3 groups chosen from halogen, CF₃, C₁₋₄alkyl and CN.

Examples of five-membered aromatic rings containing 1, 2 or 3 heteroatoms selected from N, O and S include pyrrolyl, pyrazolyl, imidazolyl, triazolyl, furanyl, thienyl, oxazolyl, isoxazolyl, thiazolyl, isothiazolyl, oxadiazolyl and thiadiazolyl. Favoured five-membered rings are thienyl and thiazolyl.

Examples of six-membered aromatic rings containing 1, 2 or 3 heteroatoms selected from N, O and S include pyridyl, pyrazinyl, pyrimidinyl, pyridazinyl, triazine and parathiazinyl. Favoured six-membered rings are pyridyl and pyrimidinyl.

Preferably, R⁴ is unsubstituted or substituted by 1 or 2 groups selected from halogen, CF₃, methyl, ethyl and CN.

R⁵ is preferably hydrogen, C₁₋₆alkyl, aryl, S(O)₂R^{g}, C(O)OR^{g} or C(O)R^{g}, where R^{g} is as hereinbefore defined. More preferably, R⁵ is hydrogen, C₁₋₄alkyl, phenyl, S(O)₂R^{g} or C(O)R^{g}, where R^{g} is C₁₋₆alkyl or heteroaryl. Most preferably, R⁵ is hydrogen, methoxycarbonyl, ethyl, methyl, phenyl, S(O)₂CH₃, C(O)CH₃,

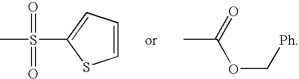

When R⁴ and R⁵ form a mono- or bicyclic ring together with the nitrogen atom to which they are attached, it preferably contains from 5 to 8 carbon atoms, such as azepanyl or hexahydrocyclopenta[c]pyrrol-2(1H)-yl.

X and Y are preferably independently hydrogen or methyl, most preferably hydrogen.

In one embodiment of the present invention, there is provided the compound of formula (Ia): 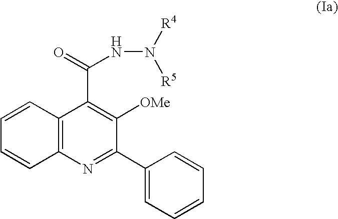
or a pharmaceutically acceptable salt thereof,
wherein R⁴ is as defined in relation to formula (I), and R⁵ is C(O)NR^{g}R^{h}, S(O)NR^{g}R^{h}, S(O)₂R^{g}, C(O)OPh, C(O)O CH₂Ph or C(O)R^{g}, where R^{g} is as defined in relation to formula (I).

Preferably, R⁴ is phenyl, pyridyl, pyrimidinyl or benzothiazolyl, optionally substituted by 1 or 2 groups chosen from halogen, CF₃, methyl, ethyl and CN.

Preferably, R⁵ is S(O)₂R^{g}, C(O)OCH₂Ph or C(O)R^{g}, where R^{g} is selected from methyl, ethyl, iso-propyl, benzyl, (CH₂)₀₋₁heteroaryl, phenyl, (CH₂)₀₋₁C₃₋₈cycloalkyl and (CH₂)₀₋₁Het, optionally substituted by 1 to 5 fluorine atoms.

In another embodiment of the present invention, there is provided the compound of formula (Ib): 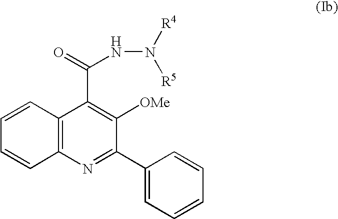
or a pharmaceutically acceptable salt thereof,
wherein R⁴ is C₁₋₈alkyl, C₂₋₈alkenyl, C₂₋₈alkynyl, C₃₋₈cycloalkyl, aryl or arylC₁₋₆alkyl, substituted by hydroxyl or 1 to 8 halogen atoms,
or R⁴ is a moiety containing at least one aromatic ring and possesses 5, 6, 9 or 10 ring atoms of which 1, 2, 3 or 4 atoms are heteroatoms independently selected from N, O and S, which ring system is optionally substituted at any substitutable position by 1, 2 or 3 groups chosen from hydroxy, halogen, NO₂, CN, NH₂, C₁₋₄alkyl, C₂₋₄alkenyl, C₂₋₄alkynyl, C₁₋₄alkoxy and C(O)OC₁₋₆alkyl, which group is optionally substituted by 1 to 8 halogen atoms; and
R⁵ is as defined in relation to formula (I).

Preferably, R⁴ is phenyl substituted by 1 or 2 groups chosen from halogen, CF₃, OCF₃, methyl, ethyl and CN,

or R⁴ is pyridyl, pyrimidinyl or benzothiazolyl, optionally substituted by 1 or 2 groups chosen from halogen, C₁₋₄alkyl, C₁₋₄alkoxy, CF₃, OCF₃ and NH₂.

Preferably, R⁵ is hydrogen, C₁₋₆ alkyl, aryl, S(O)₂R^{g}, C(O)OR^{g} or (O)R^{g}, where R^{g} is as defined in relation to formula (I). More preferably, R⁵ is S(O)₂R^{g}, C(O)OCH₂Ph or C(O)R^{g}, where R^{g} is selected from methyl, ethyl, iso-propyl, (CH₂)₀₋₁phenyl, (CH₂)₀₋₁C₃₋₈cycloalkyl, (CH₂)₀₋₁-heteroaryl and (CH₂)₀₋₁Het, optionally substituted by 1 to 5 fluorine atoms.

In another embodiment of the present invention, there is provided the compound of formula (Ic): 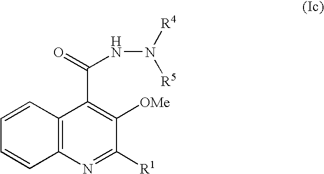
or a pharmaceutically acceptable salt thereof,
wherein R¹ is a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a},
where R^{a} is as defined in relation to formula (I), and
R⁴ and R⁵ are as defined in relation to formula (I).

Preferably, R¹ is Het. More preferably, R¹ is 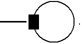
Most preferably, R¹ is pyrrolidinyl, piperidinyl or piperazinyl.

Preferably, R⁴ is phenyl, pyridyl, pyrimidinyl or benzothiazolyl, optionally substituted by 1 or 2 groups chosen from halogen, CF₃, methyl, ethyl and CN.

Preferably, R⁵ is hydrogen, C₁₋₆ alkyl, aryl, S(O)₂R^{g}, C(O)OR^{g} or C(O)R^{g}, where R^{g} is as defined in relation to formula (I). More preferably, R⁵ is S(O)₂R^{g}, C(O)OCH₂Ph or C(O)R^{g}, where R^{g} is selected from methyl, ethyl, iso-propyl, (CH₂)₀₋₁-phenyl, (CH₂)₀₋₁C₃₋₈cycloalkyl, (CH₂)₀₋₁heteroaryl and (CH₂)₀₋₁Het, optionally substituted by 1 to 5 fluorine atoms.

In another embodiment of the present invention, there is provided the compound of formula (Id): 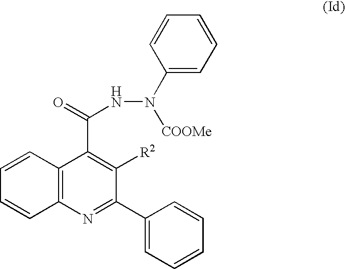
or a pharmaceutically acceptable salt thereof, wherein R² is as defined in relation to formula (I).

Preferably, R² is (CH₂)₀₋₆NR^{c}R^{d} or C₁₋₆alkoxy substituted by NR^{c}R^{d} where R^{c} and R^{d} are as defined in relation to formula (I). More preferably, R² is CH₂NR^{c}R^{d} or OCH₂CH₂NR^{c}R^{d} where R^{c} and R^{d} are as defined in relation to formula (I). Examples of suitable NR^{c}R^{d} groups are N(CH₃)₂, N(CH₃)(cyclohexyl) and morpholinyl.

The independent syntheses of any optical isomers or their chromatographic separations may be achieved as known in the art. Their absolute stereochemistry may be determined by the x-ray crystallography of crystalline products or crystalline intermediates which are derivatized, if necessary, with a reagent containing an asymmetric center of known absolute configuration.

As used herein, the term “C₁₋₈alkyl” means linear or branched chain alkyl groups having from 1 to 8 carbon atoms and includes all of the octyl, heptyl, hexyl and pentyl alkyl isomers as well as n-, iso-, sec- and t-butyl, n- and isopropyl, ethyl and methyl. “C₁₋₆alkyl”, “C₁₋₄alkyl” and “C₁₋₂alkyl” shall be understood in an analogous manner, as shall “C₁₋₆ alkoxy” and “C₁₋₄ alkoxy”.

The term “C₂₋₈alkenyl” means linear or branched chain alkenyl groups having from 2 to 8 carbon atoms and includes all of the hexenyl and pentenyl isomers as well as 1-butenyl, 2-butenyl, 3-butenyl, isobutenyl, 1-propenyl, 2-propenyl, and ethenyl (or vinyl).

The term “C₂₋₈alkynyl” means linear or branched chain alkynyl groups having from 2 to 8 carbon atoms and includes all of the octynyl, heptynyl, hexynyl and pentynyl isomers as well as 1-butynyl, 2-butynyl, 3-butynyl, 1-propynyl, 2-propynyl, and ethynyl (or acetylenyl).

The term “C₃₋₈cycloalkyl” means a cyclic alkane ring having three to eight total carbon atoms (i.e., cyclopropyl, cyclobutyl, cyclopentyl, cyclohexyl, cycloheptyl, or cyclooctyl). The term “C₄₋₇cycloalkyl” refers to a cyclic ring selected from cyclobutyl, cyclopentyl, cyclohexyl and cycloheptyl.

The term “halogen” refers to fluorine, chlorine, bromine and iodine.

The term “heteroaryl” as used herein is intended to include the following groups: furanyl, imidazolyl, isothiazolyl, isoxazolyl, oxadiazolyl, oxazolyl, pyrazinyl, pyrazolyl, pyridazinyl, pyridazinyl, pyridyl, pyrimidyl, pyrrolyl, tetrazolyl, thiadiazolyl, thiazolyl, thienyl, triazolyl and pyrrolidinyl.

The term “Het” means a heteroaliphatic ring of 3 to 7 ring atoms, which ring contains 1, 2 or 3 heteroatoms selected from N, O or S or a group S(O), S(O)₂, NH or NC₁₋₄alkyl. Examples of Het include aziridinyl, azetidinyl, pyrrolidinyl, piperidinyl, azepinyl, piperazinyl, imidazolidinyl, pyrazolidinyl, tetrahydrofuranyl, dioxolanyl, pyran, dioxanyl, morpholine, oxathiolanyl, dithianyl, oxathianyl, thiomorpholinyl, trioxanyl, trithianyl.

The terms “thiophenyl” and “thienyl” have the same meaning herein and are used interchangeably. Similarly, the following pair of terms has the same meaning: “pyridinyl” and “pyridyl”.

Exemplary compounds of the present invention include:
methyl 1-(2-fluorophenyl)-2-[(3-methoxy-2-phenylquinolin-4-yl)carbonyl]hydrazinecarboxylate,
methyl 1-(3-fluorophenyl)-2-[(3-methoxy-2-phenylquinolin-4-yl)carbonyl]hydrazinecarboxylate,
methyl 1-(4-fluorophenyl)-2-[(3-methoxy-2-phenylquinolin-4-yl)carbonyl]hydrazinecarboxylate,
methyl 1-(2-chlorophenyl)-2-[(3-methoxy-2-phenylquinolin-4-yl)carbonyl]hydrazinecarboxylate,
methyl 1-(3-chlorophenyl)-2-[(3-methoxy-2-phenylquinolin-4-yl)carbonyl]hydrazinecarboxylate,
methyl 1-(4-chlorophenyl)-2-[(3-methoxy-2-phenylquinolin-4-yl)carbonyl]hydrazinecarboxylate,
methyl 2-[(3-methoxy-2-phenylquinolin-4-yl)carbonyl]-1-(2-methylphenyl)hydrazinecarboxylate,
methyl 2-[(3-methoxy-2-phenylquinolin-4-yl)carbonyl]-1-(3-methylphenyl)hydrazinecarboxylate,
methyl 2-[(3-methoxy-2-phenylquinolin-4-yl)carbonyl]-1-(4-methylphenyl)hydrazinecarboxylate,
methyl 1-(2-ethylphenyl)-2-[(3-methoxy-2-phenylquinolin-4-yl)carbonyl]hydrazinecarboxylate,
methyl 2-[(3-methoxy-2-phenylquinolin-4-yl)carbonyl]-1-[3-(trifluoromethyl)phenyl]hydrazinecarboxylate,
methyl 1-(4-bromophenyl)-2-[(3-methoxy-2-phenylquinolin-4-yl)carbonyl]hydrazinecarboxylate,
methyl 1-(4-iodophenyl)-2-[(3-methoxy-2-phenylquinolin-4-yl)carbonyl]hydrazinecarboxylate,
methyl 1-(4-cyanophenyl)-2-[(3-methoxy-2-phenylquinolin-4-yl)carbonyl]hydrazinecarboxylate,
methyl 2-[(3-methoxy-2-phenylquinolin-4-yl)carbonyl]-1-[4-(trifluoromethoxy)phenyl]hydrazinecarboxylate,
methyl 1-(2,5-difluorophenyl)-2-[(3-methoxy-2-phenylquinolin-4-yl)carbonyl]hydrazinecarboxylate,
methyl 1-(3-chloro-4-fluorophenyl)-2-[(3-methoxy-2-phenylquinolin-4-yl)carbonyl]hydrazinecarboxylate,
methyl 1-(2,6-dichlorophenyl)-2-[(3-methoxy-2-phenylquinolin-4-yl)carbonyl]hydrazinecarboxylate,
methyl 1-(3,5-dichlorophenyl)-2-[(3-methoxy-2-phenylquinolin-4-yl)carbonyl]hydrazinecarboxylate,
methyl 1-(2,5-dichlorophenyl)-2-[(3-methoxy-2-phenylquinolin-4-yl)carbonyl]hydrazinecarboxylate,
methyl 1-(3-chloro-4-methylphenyl)-2-[(3-methoxy-2-phenylquinolin-4-yl)carbonyl]hydrazinecarboxylate,
methyl 1-[2-chloro-5-(trifluoromethyl)phenyl]-2-[(3-methoxy-2-phenylquinolin-4-yl)carbonyl]hydrazinecarboxylate,
methyl 2-[(3-methoxy-2-phenylquinolin-4-yl)carbonyl]-1-pyridin-2-ylhydrazinecarboxylate,
methyl 2-[(3-methoxy-2-phenylquinolin-4-yl)carbonyl]-1-[4-(trifluoromethyl)pyrimidin-2-yl]hydrazinecarboxylate,
methyl 1-(1,3-benzothiazol-2-yl)-2-[(3-methoxy-2-phenylquinolin-4-yl)carbonyl]hydrazinecarboxylate,
   and their pharmaceutically acceptable salts.

Further exemplary compounds of the present invention include:
benzyl 2-[(3-methoxy-2-phenylquinolin-4-yl)carbonyl]-1-phenylbydrazinecarboxylate,
N′-acetyl-3-methoxy-N′,2-diphenylquinoline-4-carbohydrazide,
N′-isobutyryl-3-methoxy-N′,2-diphenylquinoline-4-carbohydrazide,
N′-(cyclopentylacetyl)-3-methoxy-N′,2-diphenylquinoline-4-carbohydrazide,
N′-(4-fluorobenzoyl)-3-methoxy-N′,2-diphenylquinoline-4-carbohydrazide,
N′-2-furoyl-3-methoxy-N′,2-diphenylquinoline-4-carbohydrazide,
3-methoxy-N′,2-diphenyl-N′-(phenylacetyl)quinoline-4-carbohydrazide,
3-methoxy-N′,2-diphenyl-N′-(2-thienylacetyl)quinoline-4-carbohydrazide,
3-methoxy-N′-(methylsulfonyl)-N′,2-diphenylquinoline-4-carbohydrazide,
3-methoxy-N′,2-diphenyl-N′-(2-thienylsulfonyl)quinoline-4-carbohydrazide,
3-methoxy-N′-(morpholin-4-ylcarbonyl)-N′,2-diphenylquinoline-4-carbohydrazide,
methyl 2-[(3-methyl-2-pyrrolidin-1-ylquinolin-4-yl)carbonyl]-1-phenylhydrazinecarboxylate,
   and their pharmaceutically acceptable salts.

Additional exemplary compounds of the present invention include
3-[2-(dimethylamino)ethoxy]-2-phenyl-4-quinolinecarboxylic acid, 2-methoxycarbonyl)-2-phenylhydrazide,
3-[2-(4-morpholinyl)ethoxy]-2-phenyl-4-quinolinecarboxylic acid, 2-(methoxycarbonyl)-2-phenylhydrazide,
3-(4-morpholinylmethyl)-2-phenyl-4-quinolinecarboxylic acid, 2-(methoxycarbonyl)-2-phenylhydrazide,
3-[(cyclohexylmethylamino)methyl]-2-phenyl-4-quinolinecarboxylic acid, 2-(methoxycarbonyl)-2-phenylhydrazide,
   and their pharmaceutically acceptable salts.

These compounds and those defined by the immediately preceding definitions are useful in therapy, especially as NK-2 and/or NK-3 antagonists, particularly as NK-3 antagonists.

The terms “administration of” and or “administering a” compound should be understood to mean providing a compound of the invention to the individual in need of treatment.

The term “subject,” (alternatively referred to herein as “patient”) as used herein refers to an animal, preferably a mammal, most preferably a human, who has been the object of treatment, observation or experiment.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, pamoate, and the like, can be used as the dosage form.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

The compounds of the present invention may be administered by oral, parenteral (e.g. intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require NK-3 receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention also provides pharmaceutical compositions comprising a compound of formula (I) or a pharmaceutically acceptable salt thereof and a pharmaceutically acceptable excipient.

Thus, there is provided a compound of formula (I) or a pharmaceutically acceptable salt thereof for use in a method of treatment of the human or animal body by therapy.

Likewise, there is provided the use of a compound of formula (I) for the manufacture of a medicament for treating a neurokinin-2 and/or neurokinin-3 mediated disease.

There is also disclosed a method of treatment of a subject suffering from a neurokinin-2 and/or neurokinin-3 mediated disease, which comprises administering to that patient a therapeutically effective amount of a compound of formula (I) or a pharmaceutically acceptable salt thereof.

Examples of diseases mediated by neurokinin-2 and/or neurokinin 3 include CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform-diseases, -acute psychosis, alcohol psychosis, autism, delirium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophiclateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased, sensitivity, to noxious stimuli (thermal, cold, mechanical hyperalgesia), continuing pain sensation after removal of the stimulation (hyperpathia) or an absence of or deficit in selective sensory pathways (hypoalgesia), pain associated with migraine, and non-cardiac chest pain); certain CNS-mediated disorders such as emesis, irritable bowel syndrome, and non-ulcer dyspepsia; COPD, asthma, cough, gastro-oesophageal reflex induced cough, and exacerbated asthma; urinary incontinence; hypertension; and conditions associated with platelet hyperaggregability such as tissue ulceration, nephrotic syndrome, diabetes, migraine, coronary artery disease, pre-eclampsia and stroke. Preferably, the compounds of the invention are useful for the treatment of depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; and irritable bowel syndrome. More preferably, the compounds of the invention are useful for the treatment of depression; anxiety disorders; phobias; and psychosis and psychotic disorders (especially schizophrenia, schizo-affective disorder, and schizophreniform diseases. Most preferably, the compounds of the invention are useful for the treatment of schizophrenia.

The compounds for use in the present invention are generally active in the following tests. They normally have an IC₅₀ of less than 1 μM and preferably less than 100 nM.

Details of the NK-2 receptor and its heterologous expression can be found in Gerard et al., J. Biol. Chem., 265: 20455-20462, 1990 and Huang et al., Biochem., 33: 3007-3013, 1994. The latter paper also contains details of mutant scanning.

Details of the NK-3 receptor and its heterologous expression can be found in Huang et al., BBRC, 1992, 184: 966-972 and Sadowski et al., Neuropeptides, 1993, 24: 317-319.

A membrane preparation is prepared as follows. A 10-layer cell factory is seeded with CHO cells stably expressing NK-3 receptors. The CHO cells are prepared in a triple T175 flask in 11 growth medium which contains Iscore's modified Dulbecco's medium containing 10 ml/l 200 mM L-Glutamine, 10 ml/l penicillin-streptomycin, one vial of hypoxanthine-thymidine 500x/l, 1 mg/ml geneticin and 10% fetal bovine serum (inactivated). The cells are grown for 3 days in an incubator. The medium is washed off and the factory is rinsed twice with 400 ml PBS (Ca, Mg-free). 400 ml enzyme free dissoc. solution (EFDS) is added and the factory is maintained for 10 min at room temperature. The cells are dislodged and the suspension poured into 500 ml centrifuge bottles. The process is repeated with 200 ml EFDS and the mixtures pooled giving 6 bottles in all, which are spun in a centrifuge for 10 min at 2200 rpm.

The supernatants are aspirated and the residual cell pellets are frozen at −80° for 30 min to improve cell lysis and then resuspended in 40 ml Tris with inhibitors per cell factory. The cells are homogenized in 40 ml aliquots with 8 strokes of a glass-teflon grinder at setting 40. The homogenate is transferred to 50 ml centrifuge tubes and placed on a rocker for 15 min at r.t. The homogenate is rehomogenised and held on ice if necessary before being centrifuged again as above.

The supernatant is transferred to Sorvall tubes for an SS-34 roter and held on ice.

40 ml cold Tris with inhibitors is used to resuspend and combine the pellets which are again spun as above. The supernatants are again transferred to Sorvall tubes which, with those above, are spun at 18000 rpm for 20 min.

The supernatants are discarded and the pellets resuspended in a Storage Buffer consisting of 2.50 ml 1M Tris pH7.4, 50 μl 1000× protease inhibitors (4 mg/ml leupeptin (Sigmo), 40 mg/ml Bacitracin (Sigma) and 10 mM phosphoranidon (Peninsula) all dissolved in water) plus 0.5 ml 0.5M MnCl₂ made up to 50 ml with H₂O_{dd}. A 10 ml syringe is used with 20-, 23- and 25-gauge needles sequentially.

A Bradford protein assay in conducted on 2-10 μl aliquots with BSA as standard before 500-1000 μl aliquots are snap-frozen in liquid nitrogen for storage at −80° C.

The membrane binding assay is carried out as follows. The amount of membranes needed to specifically bind ≦10% of ¹²⁵I-NeurokinB is predetermined. The frozen stocks are then diluted to allow addition in 50 μl.

The test compounds are dissolved in DMSO. An automated apparatus (Tecan) is programmed to add 5 μl of compound or DMSO, approximately 100,000 cpm of isotope in 20 μl buffer which is prepared from 50 μMTris, pH7.5, 150 μM NaCl, bovine serum albumin to 0.02%, and protease inhibitors as in the storage buffer, made up as 0.5M stock, and 175 μl assay buffer (as the storage buffer but containing 5 μM MnCl₂ and without NaCl) into deep well Marsh boxes (Marsh Biomedical Products) in a 96-well format. Excess unlabelled competing peptide is added by hand for non-specific binding as indicated below. The binding reaction is initiated by adding 50 μl of cell membranes. The tubes are incubated with shaking for 1 h at r.t and filtered on a Tomtec 96 well cell harvester using Mach III filtermats (Tomtec) or using either a Packard 96-well harvester or Tomtec 9600 using Unifilter GF/C (Packard), presoaked in 0.25% polyethyleneimine and washed five times with 1× wash buffer (0.1 M.Tris, pH7.4 and 1M NaCl, 1×=100ml of 10× stock per litre of cold distilled water). If using Unifilter plates, 60 μl Microscint 20 (Packard) is added to each well and the plate is then heat-sealed before counting in a Packard Topcount. Alternatively the filters from the filtermat are placed in 75×100 mm plastic tubes and counted on a Cobra gamma counter.

For the assay, typically 10 kg of membrane is used at 25,000 cpm which is filtered over a Unifilter GF/C presoaked in 0.5% BSA.

Assays for binding at the neurokinin-2 receptor can be carried out in an analogous manner.

Abbreviations used in the instant specification, particularly the Schemes, Descriptions and Examples, include the following:

Ac=acetyl; 9-BBN=9-borabicyclo[3.3.1]nonane; Bn=benzyl; BOC or Boc=t-butyloxycarbonyl; Bu=butyl; t-Bu=tert-butyl; CBZ=carbobenzoxy (alternatively, benzyloxycarbonyl); CDI=carbonyl diimidazole; DAST=(diethylamino)sulfur trifluoride; DCC=dicyclohexyl carbodiimide; DCM=dichloromethane; DIBAL=diisobutylaluminum hydride; DIEA or DIPEA=diisopropylethylamine; DIAD=diisopropylazodicarboxylate; DMA=N,N-dimethylacetamide; DMF=N,N-dimethylformamide; DMSO=dimethylsulfoxide; EDAC=1-(3-dimethylamino)propyl-3-ethylcarbodiimide; Et=ethyl; ether=diethyl ether; EtOAc=ethyl acetate; h=hour(s); HMDS=hexamethyldisilazyl; HOBT or HOBt=1-hydroxy benzotriazole hydrate; KHMDS=potassium hexamethyldisilazide; LDA=lithium diisopropylamide; Me=methyl; m=minute(s); Ph=phenyl; Pr=propyl; i-Pr=isopropyl; PMB=p-methoxybenzyl; PS=polymer-supported; sat'd=saturated aqueous; rt=room temperature; TBSO=t-butyldimethylsiloxy; TEA=triethylamine; Tf=triflic or triflate; TFA=trifluoroacetic acid; THF=tetrahydrofuran; TPAP=tetrapropylammonium perruthenate.

Compounds of formula I can be made by reacting a compound of formula (II) with a compound of formula (III): 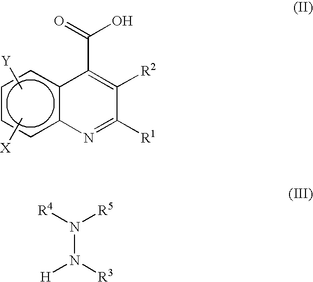
wherein R¹, R², R³, R⁴, R⁵, X and Y are as defined above. The reaction is generally carried out in a solvent such as CH₂Cl₂ and/or THF in the presence of a base such as Et₃N and a condensing agent such as HBTU or HOBT with EDC.HCl. Depending on the precise nature of the reagents, reaction conditions can range from 1 to 18 hours and from 0° C. to reflux.

If necessary the compound of formula (II) can be converted into its acid chloride prior to reacting with the compound of formula (III); this can be done by reacting with oxalyl chloride for about 18 hours at room temperature.

If desired compounds of formula I can be converted into other compounds of formula I, particularly when it is desired to transform one group R⁵ into another, by means known in the art. For example, compounds in which R⁵ is H can be converted into compounds where R⁵ is methoxycarbonyl by reacting with methyl chloroformate optionally in the presence of a base such as Et₃N and a solvent such as CH₂Cl₂ for about 4 hours at room temperature.

Compounds of formulae (II) and (III) are generally known in the art or can be produced from known compounds by methods known in the art. For example, compounds of formula (II) can be made by reacting compounds of formulae (IV) and (V): 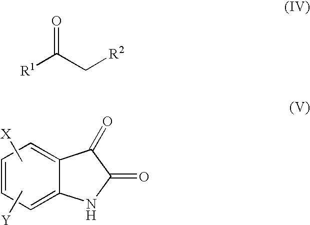
wherein R¹, R², X and Y are as defined above. The reaction is generally carried out in the presence of a strong base such as potassium hydroxide, a solvent such as ethanol and at reflux.

Compounds of formulae (IV) and (V) are known in the art or can be made by known methods from known compounds.

Compounds of formula (II) in which R² is C₁₋₆alkylNR′R″ can be made by reacting compounds of formula (II) in which R² is C₁₋₆alkyl successively with N-bromosuccinimide (generally in a solvent such as CH₃CN, at reflux and with an initiation agent such as dibenzoylperoxide) and then HNR′R″ (generally in the presence of a base such as diisopropylethylamine, in a solvent such as THF at about 50° C.). During this procedure, the carboxyl group of the compound of formula (I) may be protected as the methyl ester.

Components of formula HNR′R″ are known in the art or can be made by known methods from known compounds.

The compounds of the present invention can be readily prepared according to the following reaction schemes and examples, or modifications thereof. Starting materials can be made from procedures known in the art or as illustrated. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, the variables are as defined above.

Compounds of formula (I) where R² is an O-linked group can be made by the general method shown in Scheme 1:

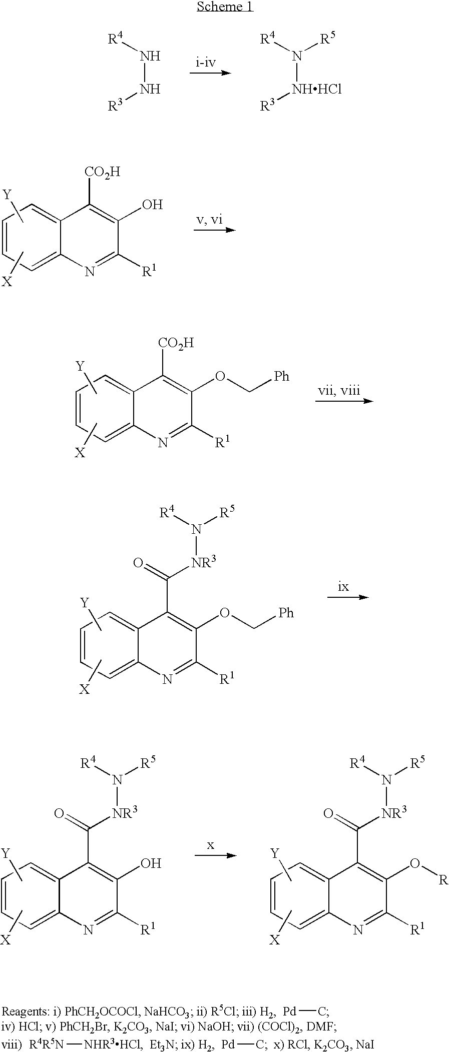

Compounds of formula (I) where R² is CH₂NR^{c}R^{d} can be made by the general method shown in Scheme 2:

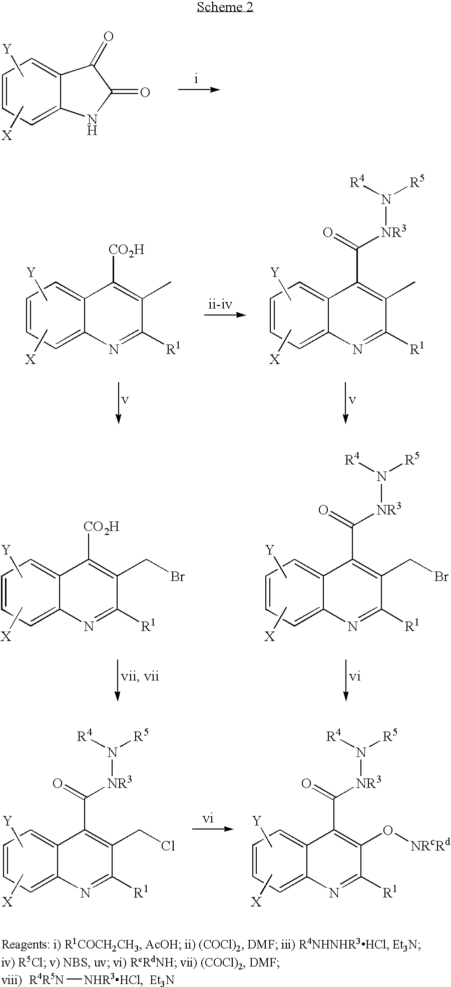

¹H nmr spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are reported in the order: number of protons; multiplicity (s, singlet; d, doublet; t, triplet; q, quartet; m, multiplet; br, broad; and combinations thereof); coupling constant(s) in hertz. Mass spectral (MS) data were obtained on a Waters Micromass ZQ or a Waters Micromass ZMD operating in negative (ES⁻) or positive (ES⁺) ionisation mode and results are reported as the ratio of mass over charge (m/z) for the parent ion only. Preparative scale HPLC separations were carried out using mass triggered HPLC on a preparative Agilent 100 separation module. Compounds were either eluted with linear gradients of acetonitrile/0.1% TFA and water/0.1% TFA or with acetonitrile and water (containing ammonium carbonate to give a pH of 10). In all cases flow rates between 15 and 25 mL/min were used.

The following Descriptions and Examples further illustrate the present invention:

### Description 1: Phenylmethyl 2-Phenylhydrazinecarboxylate

To a stirred cooled (5° C.) solution of phenyl hydrazine (20 g, 0.185 mol) in EtOAc and saturated aqueous NaHCO₃ (200 mL) was added K₂CO₃ (10 g, 0.72 mol) followed by the slow addition (over 0.5 h.) of a solution of benzyl chloroformate (34.8 g, 0.204 mol) in EtOAc (100 mL). The mixture was stirred at 0° C. to 5° C. for 0.5 h., the layers were separated, the organic phase was dried (MgSO₄) and the solvent was evaporated under reduced pressure. The residue was washed with isohexane (2×100 mL) and dried in vacuo to give 43.9 g of the title compound.

¹H NMR (CDCl₃, 500Mz) δ 7.42-7.05 (11H, m), 5.23 (2H, m), 3.79 (3H, m) as a mixture of rotamers.

### Description 2: 1-Methyl 2-Phenylmethyl 1-Phenyl-1,2-hydrazinedicarboxylate

To a suspension of the product of Description 1 in toluene (500 mL) was added methyl chloroformate (17.5 mL, 0.226 mol) and the mixture was stirred at 100° C. for 1.5 h. The resulting clear solution was cooled to rt and the solvent was evaporated under reduced pressure to give the title compound as a foam (58 g).

### Description 3: Methyl 1-Phenylhydrazinecarboxylate Hydrochloride

A mixture of the product of Description 2 (58 g) and 10% Pd on C (4 g) in methanol (400 mL) was hydrogenated for 1 h. at 30-40 psi of hydrogen. The mixture was filtered and methanolic hydrogen chloride (1M, 200 mL) was added. The solvent was evaporated under reduced pressure and the solid residue was washed with ether to give 31.9 g of the title compound. ¹H NMR (CDCl₃, 500Mz) δ 9.2 (3H, broad s), 7.55 (2H, d, J 7.5), 7.45 (2H, t, J 6.7), 7.35 (1H, t, J 7.5), 3.73 (3H, s) as a mixture of rotamers.

### Description 4: 2-Phenyl-3-(phenylmethoxy)quinoline-4-carboxylic Acid

To a suspension of 3-hydroxy-2-phenylquinoline-4-carboxylic acid (2.65 g, 0.01 mol, as described in Giardina et al., J. Med. Chem. 1999, 42, 1053-1065) and K₂CO₃ (5.53 g, 0.04 mol) in THF (50 mL) was added benzyl bromide (2.99 mL, 0.025 mol) and sodium iodide (0.01 g) and the mixture was heated under reflux for 14 h. The mixture was cooled, the solvent was partially evaporated under reduced pressure to ca. 20 mL, a further portion of benzyl bromide (1 mL, 0.008 mol) was added and the mixture was heated under reflux for 24 h. The mixture was cooled, filtered and the solvent was evaporated under reduced pressure. The residue was dissolved in methanol (100 mL), aqueous NaOH (2N, 25 mL) was added and the mixture was heated under reflux for 4 h. The mixture was cooled, the solvents were evaporated under reduced pressure and the residue was partitioned between water (100 mL) and ether (2×100 mL). The aqueous layer was acidified with concentrated hydrochloric acid and the solid produced was collected by filtration, washed with water and ether and dried in vacuo at 80° C. to give 2.45 g of the title compound as a colorless solid.

¹H NMR (500 MHz, DMSO-d6): δ 14.30 (br, s, 1H), 8.12 (d, 1H), 8.00 (dd, 2H), 7.85-7.79 (m, 2H), 7.71 (t, 3H), 7.55 (t, 3H), 7.36-7.34 (m, 3H), 7.18-7.16 (m, 2H), 4.68 (s, 2H). m/z (ES⁺) 356 [M+H⁺]

### Description 5: 2-Phenyl-3-(phenylmethoxy)-4-quinolinecarboxylic acid, 2-(Methoxycarbonyl)-2-phenylhydrazide

To a solution of 2-phenyl-3-(phenylmethoxy)quinoline-4-carboxylic acid (Description 4, 2.12 g, 6 mmol) in DCM (60 mL) was added oxalyl chloride (0.782 mL, 9 mmol) and 1 drop of DMF. The solution was stirred at rt for 1 h., then the solvent was evaporated under reduced pressure. Two further portions of DCM (50 mL) were added and evaporated under reduced pressure. The residue was redissolved in DCM (50 mL) and added to a solution containing methyl 1-phenylhydrazinecarboxylate hydrochloride (Description 3, 1.8 g, 9 mmol) and TEA (3.3 mL, 24 mmol) in DCM (90 mL). The clear solution was stirred at rt for 14 h. and then was partitioned between DCM and saturated aqueous NaHCO₃. The organic phase was dried (Na₂SO₄) and evaporated to a small volume. The solution was applied to a column containing silica gel and the product eluted with increasing concentrations (0% to 30%) EtOAc in isohexane to give the title compound as a foam (2.45 g).

¹H NMR (500 MHz, CDCl₃): δ 8.26 (2H, s), 8.17 (1H, d, J 8.3), 7.99 (2H, s), 7.72 (1H, t, J 7.1), 7.63 (1H, t, J 7.2), 7.49 (5H, s), 7.19 (2H, t, J 7.3), 6.92 (2H, d, J 7.2), 4.66 (2H, s), 3.88 (3H, s). m/z (ES⁺) 504 [M+H⁺].

### Description 6: 3-Hydroxy-2-phenyl-4-quinolinecarboxylic acid, 2-(Methoxycarbonyl)-2-phenylhydrazide

To a solution of 2-phenyl-3-(phenylmethoxy)-4-quinolinecarboxylic acid, 2-(methoxycarbonyl)-2-phenylhydrazide (Description 5, 2.4 g, 0.048 mmol) in methanol (80 mL) under an atmosphere of nitrogen was added 10% Pd on C (0.2 g). The mixture was shaken on a Parr apparatus under 50 psi of hydrogen for 18 h. The mixture was filtered and the solvent was evaporated under reduced pressure. The residue was recrystallised from isopropanol to give 0.73 g of the title compound as a colorless solid.

¹H NMR (500 MHz, CDCl3): 9.95 (1H, br s), 8.46 (1H, br s), 8.13-8.04 (4H, m), 7.60-7.42 (9H, m), 7.33 (1H, t), 3.90 (3H, s). m/z (ES⁺) 414 [M+H⁺].

### Description 7: 3-Methyl-2-phenylquinoline-4-carboxylic Acid

To a slurry of isatin (20 g, 0.136 mol) in acetic acid (370 mL) was added propiophenone (18 mL, 0.136 mol). The mixture was heated at 75° C. for 5 min., concentrated hydrochloric acid (124 mL) was added and the mixture was heated at 100° C. for 16 h. The mixture was cooled and water (800 mL) was added. The solid which formed was collected by filtration, washed with ether and dried in vacuo at 60° C. to give 15.6 g (43%) of the title compound as a brown solid.

¹HNMR (400 MHz, DMSO-d6) δ 8.08 (1H, d, J 8.3), 7.83 (2H, t, J 8.0), 7.71 (1H, t, J 7.6), 7.63 (2H, dd, J 2.0, 7.8), 7.57-7.51 (3H, m), 2.39 (3H, s). m/z (ES⁺) 264 [M+H⁺]

### Description 8: 3-Methyl-2-phenyl-4-quinolinecarboxylic acid, 2-Phenylhydrazide

To a suspension of 3-methyl-2-phenylquinoline-4-carboxylic acid (Description 7, 5.0 g, 0.019 mol) in DCM (60 mL) was added oxalyl chloride (3.32 mL, 0.038 mol) followed by DMF (2 drops). The mixture was stirred at rt for 1 h. and the solvent was evaporated under reduced pressure. To a solution of the residue in DCM (30 mL) was added TEA (5.30 mL, 0.038 mol) followed by a solution of phenylhydrazine (2.80 mL, 0.028 mol) in DCM (30 mL). The mixture was stirred at rt for 5 h., diluted with water/brine and extracted with DCM (×5). The combined organic layers were dried (MgSO₄), the solvent was evaporated under reduced pressure and the residue was purified by flash chromatography on silica gel eluting with 30% EtOAc/isohexane to give 4.0 g of the title compound (59%).

¹H NMR (400 MHz, CDCl₃) δ 8.16 (1H, d, J 8.3), 7.84 (1H, d, J 8.4), 7.76-7.70 (2H, m), 7.61-7.43 (6H, m), 7.32 (2H, dd, J 7.2, 8.5), 7.02 (3H, d, J 7.9), 6.56 (1H, d, J 4.6), 2.46 (3H, s). m/z (ES⁺) 354 [M+H⁺]

### Description 9: 3-Methyl-2-phenyl-4-quinolinecarboxylic acid, 2-(Methoxycarbonyl)-2-phenylhydrazide

To a suspension of 3-methyl-2-phenyl-4-quinolinecarboxylic acid, 2-phenylhydrazide (Description 8, 4.0 g, 0.011 mol) in toluene (120 mL) was added methyl chloroformate (1.75 mL, 0.022 mol) and the mixture was heated under reflux for 16 h. The mixture was cooled, diluted with water and extracted with EtOAc (×3). The combined organic phases were dried (MgSO₄) and the solvent was evaporated under reduced pressure to give 4.26 g of the title compound (95%).

¹H NMR (400 MHz, CDCl₃) δ 8.13 (2H, d, J 8.15), 8.01 (1H, br s), 7.71-7.65 (1H, m), 7.58-7.42 (8H, m), 7.34 (1H, t, J 7.4), 7.26 (1H, m), 7.17 (1H, m), 3.89 (3H, s), 2.42 (3H, s). m/z (ES⁺) 412 [M+H⁺]

### Description 10: 3-(Bromomethyl)-2-phenyl-4-quinolinecarboxylic acid, 2-(Methoxycarbonyl)-2-phenylhydrazide

A suspension of 3-methyl-2-phenyl-4-quinolinecarboxylic acid, 2-(methoxycarbonyl)-2-phenylhydrazide (Description 9, 0.15 g, 0.365 mmol) and N-bromosuccinimide (0.129 g, 0.725 mmol) in CCl₄ (20 mL) was illuminated by the light of a quartz lamp for 1.5 h. allowing the temperature to increase to approximately 75° C. The solvent was evaporated under reduced pressure and a solution of the residue dissolved in DCM was purified on silica gel eluting with 10% to 20% EtOAc in isohexane. The residue was crystallized from ether/isohexane (1:1) to give the title compound. m/z (ES⁺) 490/492(M+H).

### Description 11: 3-(Bromomethyl)-2-phenylquinoline-4-carboxylic acid

A suspension of 3-methyl-2-phenylquinoline-4-carboxylic acid (Description 7, 0.200 g, 0.76 mmol) and N-bromosuccinimide (0.270 g, 1.52 mmol) in 1,2-dichloroethane (20 mL) was exposed to the light of a quartz lamp for 5 h. The mixture was cooled and the solvent was evaporated under reduced pressure. The residue was dissolved in EtOAc and water and the product was extracted with EtOAc (×2). The organic layers were combined, dried (MgSO₄) and the solvent was evaporated under reduced pressure. The residue was purified by flash chromatography on silica gel eluting with EtOAc/isohexane/AcOH (70:30:1) to give the title compound contaminated with residual succinimide and 3-(chloromethyl)-2-phenylquinoline-4-carboxylic acid. m/z (ES⁺) 342/344 [M+H⁺]

### Description 12: 3-(Chloromethyl)-2-phenyl-4-quinolinecarboxylic acid, 2-(Methoxycarbonyl)-2-phenylhydrazide

To a suspension of 3-(bromomethyl)-2-phenylquinoline-4-carboxylic acid (Description 11, 0.100 g, 0.29 mmol) in DCM (2 mL) was added oxalyl chloride (0.051 mL, 0.58 mmol) followed by DMF (1 drop) and the mixture was stirred at rt for 1 h. The solvent was evaporated under reduced pressure, the residue was dissolved in DCM (2 mL) and TEA (0.081 mL, 0.58 mmol) followed by methyl 1-phenylhydrazinecarboxylate hydrochloride (Description 3, 0.060 g, 0.29 mmol) were added. The mixture was stirred at rt overnight, partitioned between water and DCM, extracted with DCM (×2) and the organic layers were dried (MgSO₄) and the solvent was evaporated under reduced pressure. The residue was purified by flash chromatography on silica gel eluting with isohexane/EtOAc (70:30) to give 0.043 g of the title compound (33%)

¹H NMR (400 MHz, DMSO-d6) δ8.39 (1H, s), 8.17 (1H, d, J 8.4), 7.80 (1H, t, J 7.1), 7.66-7.60 (5H, m), 7.55-7.43 (6H, m), 7.36-7.32 (1H, m), 4.79 (2H, br s), 3.92 (3H, s). m/z (ES⁺) 446/448 [M+H⁺]

### EXAMPLES 1-25

The compounds of Examples 1-25 listed in Table 1 were prepared by the following general procedure:

To a solution of 3-methoxy-2-phenylquinoline-4-carboxylic acid (Giardina et al. J. Med. Chem. 1999, 42, 1053-1065) (0.050 g, 0.18 mmol) in N,N-dimethylacetamide (1.5 mL) were added PS-carbodiimide (0.275 g, 0.36 mmol), HOBT (0.036 g, 0.27 mmol) and the appropriate hydrazine (0.18 mmol). TEA (0.025 mL, 0.18 mmol) was added when the hydrochloride salt of the hydrazine was used. The mixture was stirred under nitrogen at rt overnight. The solution was then filtered through a Si-Carbonate scavenger cartridge (6 mL, 1 g) and the solvent evaporated to dryness in a GeneVac apparatus.

The residue was dissolved in toluene (1.5 mL) and methyl chloroformate was added (0.014 ml, 0.89 mmol). The solution was heated at reflex (110° C.) overnight and then the solvent was removed in the GeneVac. The residue was purified by HPLC using a Waters X-Terra C₁₈ (30×100 mm) 5 μL column, eluting with H₂O/MeCN/0.1% TFA in a 15-100% MeCN gradient at a flow rate of 50 mL/min. HPLC retention times in Table 1 were obtained on a Phenomenex Luna CN 5 μM 4.6×50 mm column, eluting with 5-95% MeCN/H₂O+0.1% TFA at 2 mL/min.

TABLE 1

| | | | HPLC |
|---|---|---|---|
| Example | Structure | MS (ES⁺) | retention time |
| 1 | 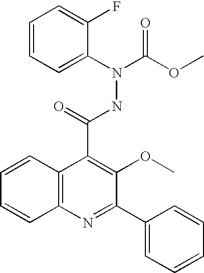 | 446 [M + H]⁺ | 3.35 |
| 2 | 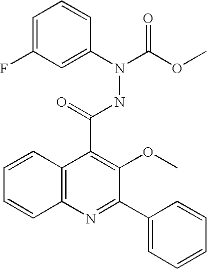 | 446 [M + H]⁺ | 3.44 |
| 3 | 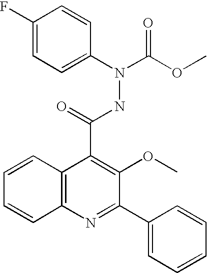 | 446 [M + H]⁺ | 3.38 |
| 4 | 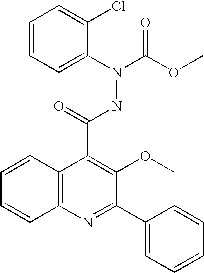 | 462/464 [M + H]⁺ | 3.45 |
| 5 | 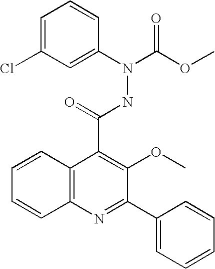 | 462/464 [M + H]⁺ | 3.58 |
| 6 | 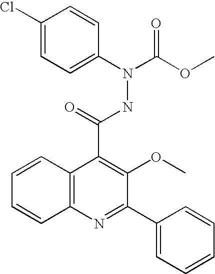 | 462/464 [M + H]⁺ | 3.56 |
| 7 | 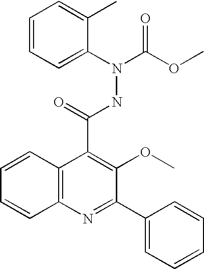 | 442 [M + H]⁺ | 3.39 |
| 8 | 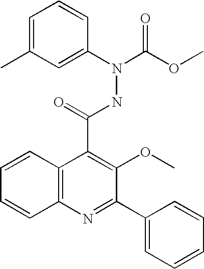 | 442 [M + H]⁺ | 3.43 |
| 9 | 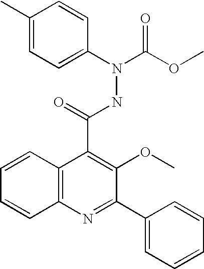 | 442 [M + H]⁺ | 3.43 |
| 10 | 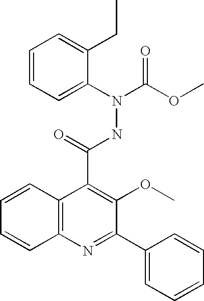 | 456 [M + H]⁺ | 3.50 |
| 11 | 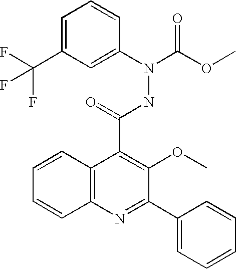 | 496 [M + H]⁺ | 3.59 |
| 12 | 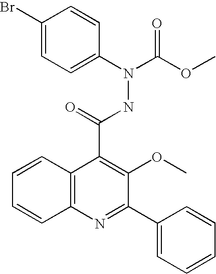 | 506/508 [M + H]⁺ | 3.59 |
| 13 | 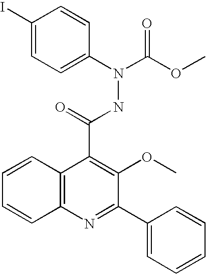 | 554 [M + H]⁺ | 3.67 |
| 14 | 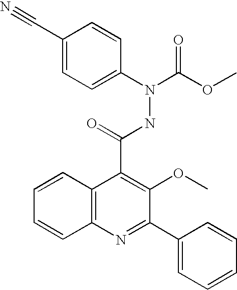 | 453 [M + H]⁺ | 3.40 |
| 15 | 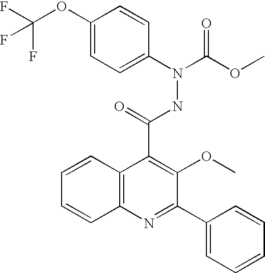 | 512 [M + H]⁺ | 3.60 |
| 16 | 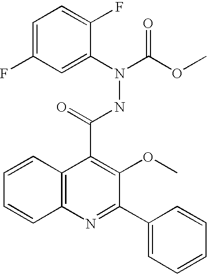 | 464 [M + H]⁺ | 3.43 |
| 17 | 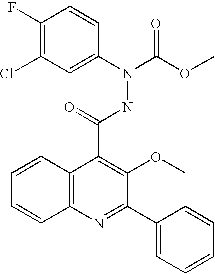 | 480/482 [M + H]⁺ | 3.59 |
| 18 | 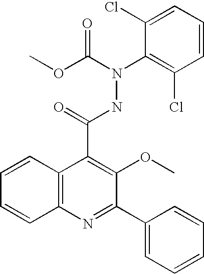 | 496/498 [M + H]⁺ | 3.43 |
| 19 | 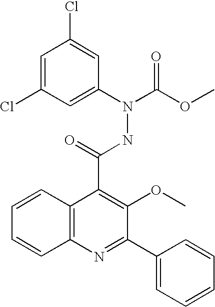 | 496/498 [M + H]⁺ | 3.81 |
| 20 | 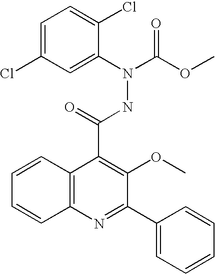 | 496/498 [M + H]⁺ | 3.64 |
| 21 | 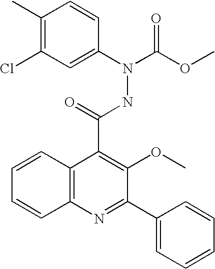 | 476/478 [M + H]⁺ | 3.43 |
| 22 | 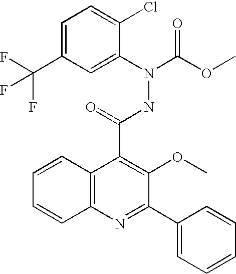 | 530/532 [M + H]⁺ | 3.66 |
| 23 | 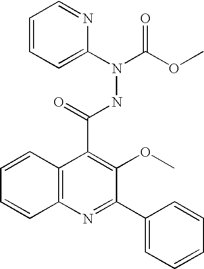 | 429 [M + H]⁺ | 3.03 |
| 24 | 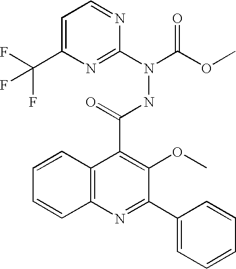 | 498 [M + H]⁺ | 3.21 |
| 25 | 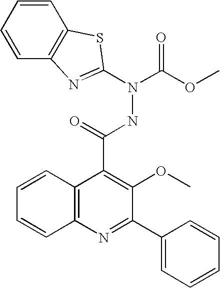 | 485 [M + H]⁺ | 3.60 |

### EXAMPLES 26-36

The compounds of Examples 26-36 listed in Table 2 were prepared by the following general procedure:

To a solution of 3-methoxy-N′,2-diphenylquinoline-4-carbohydrazide (0.040 g, 0.11) mmol) in toluene (1.5 mL) was added the appropriate chlorocarbonate/acyl chloride/sulfonyl chloride (0.22 mmol). TEA (0.030 mL, 0.22 mmol) was added when sulfonyl chloride was used. The solution was heated at reflux (110° C.) overnight and then the solvent removed in the GeneVac. The residue was purified by HPLC using a Waters X-Terra C₁₈ (30×100 mm) 5 μL column, eluting with 15-100% MeCN/H₂O+0.1% TFA gradient at a flow rate of 50 mL/min.

HPLC retention times in Table 2 were obtained on a Phenomenex Luna CN 5 μM 4.6×50 mm column, eluting with 5-95% MeCN/H₂O+0.1% TFA at 2 mL/min.

TABLE 2

| | | | HPLC |
|---|---|---|---|
| Example | Structure | MS (ES⁺) | retention time |
| 26 | 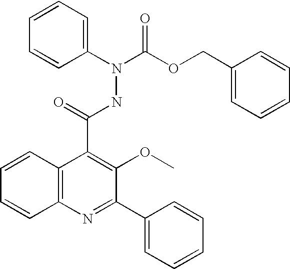 | 504 [M + H]⁺ | 3.72 |
| 27 | 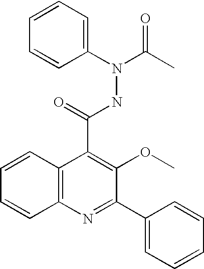 | 412 [M + H]⁺ | N/A |
| 28 | 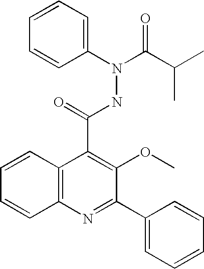 | 440 [M + H]⁺ | 3.36 |
| 29 | 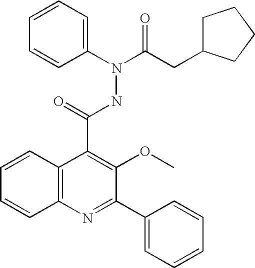 | 480 [M + H]⁺ | 3.71 |
| 30 | 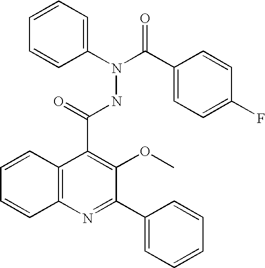 | 492 [M + H]⁺ | 3.57 |
| 31 | 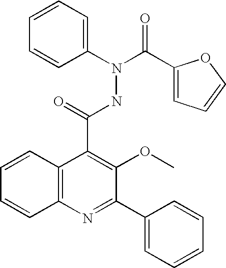 | 464 [M + H]⁺ | 3.33 |
| 32 | 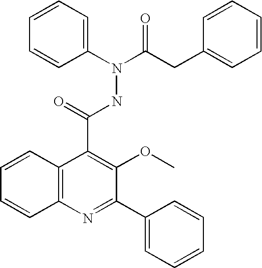 | 488 [M + H]⁺ | 3.61 |
| 33 | 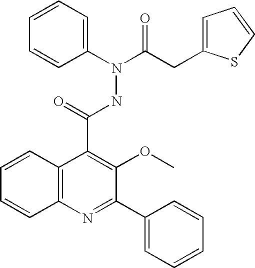 | 494 [M + H]⁺ | 3.60 |
| 34 | 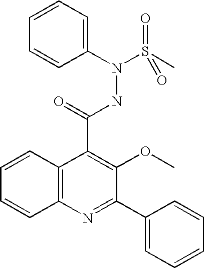 | 448 [M + H]⁺ | 3.36 |
| 35 | 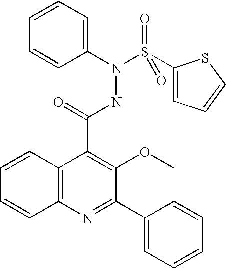 | 516 [M + H]⁺ | 3.60 |
| 36 | 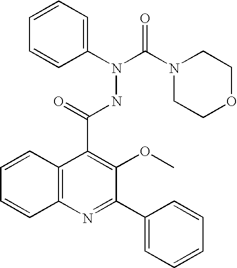 | 483 [M + H]⁺ | 3.07 |

### EXAMPLE 37

### Methyl 2-[(3-methyl-2-pyrrolidin-1-ylquinolin-4-yl)carbonyl]-1-phenylhydrazinecarboxylate

### a) 3-Methyl-2-oxo-N′phenyl-1,2-dihydroquinoline-4-carbohydrazide

To a stirred solution of 3-methyl-2-oxo-1,2-dihydroquinoline-4-carboxylic acid (R. E. Lyle et al. J. Org. Chem. 1972, 37, 3967-3968) (0.404 g, 1.99 mmol) in anhydrous DMF (20 mL) under nitrogen was added phenylhydrazine (0.235 mL, 2.39 mmol), HOBT (0.4031 g, 2.98 mmol), 1-[3-(dimethylamino)propyl]-3-ethylcarbodiimide hydrochloride (0.5719 g, 2.98 mmol) and TEA (0.416 mL, 2.98 mmol). The mixture was then stirred under nitrogen at rt for 23 h. The solvent was evaporated and water (˜20 mL) was added to the residue. The resulting solid was collected by filtration, washed with water (2×), then ether (2×), then EtOAc (2×). The solid was dried overnight in vacuum pistol at 50° C. to leave 0.1889 g (32%) of the title compound as a yellow solid. The filtrate was diluted with more EtOAc (50 mL) and water (20 mL). The organic layer was washed with aqueous citric acid (30 mL), water (30 mL), NaHCO₃ solution (30 mL) and brine (30 mL). The remaining organic layer was evaporated and the residue was purified on a silica gel column, eluting with 5-10% MeOH/CH₂Cl₂ to give another 114.5 mg (20%) of the title compound as a yellow solid: ¹H NMR (500 MHz, DMSO-d₆) δ 12.00 (1H, s), 10.44 (1H, d, J 2.9 Hz), 8.19 (1H, d, J 2.8 Hz), 7.53-7.47 (2H, m), 7.36 (1H, d, J 8.0 Hz), 7.27-7.21 (3H, m), 6.86 (2H, d, J 7.7 Hz), 6.78 (1H, t, J 7.2 Hz), 2.14 (3H, s).

### b) Methyl 2-[(3-methyl-2-oxo-1,2-dihydroquinolin-4-yl)carbonyl]-1-phenylhydrazinecarboxylate

To a stirred mixture of 3-methyl-2-oxo-N′-phenyl-1,2-dihydroquinoline-4-carbohydrazide (from Step a), 448.8 mg, 1.53 mmol) in anhydrous toluene (10 mL) under nitrogen was added methyl chloroformate (0.355 mL, 4.59 mmol) and the mixture was heated at reflux (110° C.) for 22.5 h. The mixture was cooled to rt, then filtered. The solid was washed twice with toluene and dried in a vacuum pistol at 50° C. overnight to leave 0.4605 g (86%) of the title compound as a whitish solid: ¹H NMR (500 MHz, DMSO-d₆) δ 12.01 (1H, s), 11.51 (1H, s), 7.50-7.43 (6H, m), 7.34 (1H, d, J 8.2 Hz), 7.31 (1H, t, J 6.8 Hz), 7.19 (1H, s), 3.79 (3H, s), 2.01 (3H, s).

### c) Methyl 2-[(2-bromo-3-methylquinolin-4-yl)carbonyl]-1-phenylhydrazinecarboxylate

To a stirred mixture of methyl 2-[(3-methyl-2-oxo-1,2-dihydroquinolin-4-yl)carbonyl]-1-phenylhydrazinecarboxylate (from Step b), 453.7 mg, 1.29 mmol) in anhydrous toluene (15 mL) under nitrogen was added phosphorous oxybromide (766.3 mg, 2.67 mmol) in anhydrous toluene (3+2 mL) and the mixture was heated at reflux (125° C.) for 30 min. After cooling to rt, the mixture was partitioned between EtOAc (80 mL) and water (80 mL). The aqueous layer was extracted further with EtOAc (80 mL). All organic extracts were combined, dried (Na₂SO₄) and evaporated. The residue was purified by flash chromatography on a silica gel column, eluting with 40% EtOAc/isohexane to leave 512.6 mg (96%) of the title compound as a colourless foamy solid: ¹H NMR (400 MHZ, CDCl3) δ 8.27 (1H, s), 7.99 (1H, d, J 8.4 Hz), 7.94 (1H, br s), 7.70-7.64 (1H, m), 7.59-7.55 (3H, m), 7.47-7.43 (2H, m), 7.37 (1H, t, J 7.3 Hz), 3.90 (3H, s), 2.50 (3H, s).

### d) Methyl 2-[(3-methyl-2-pyrrolidin-1-ylquinolin-4-yl)carbonyl]-1-phenylhydrazinecarboxylate

A solution of methyl 2-[(2-bromo-3-methylquinolin-4-yl)carbonyl]-1-phenylhydrazinecarboxylate (from Step c), 29.9 mg, 0.072 mmol) and pyrrolidine (0.018 mL, 0.216 mmol) in anhydrous DMA (1 mL) was heated at 120° C. for 4 min in a Smith microwave apparatus under high absorption level. (The reaction spent ˜2 min at 110-120° C.) The solvents were evaporated and the residue was purified by prep TLC (silica gel, 25% EtOAc/isohexane) to afford 17.0 mg (58%) of the title compound as a colourless solid: ¹H NMR (500 MHz, CDCl₃) δ 8.50 (1H, s), 7.67 (1H, s), 7.62-7.58 (3H, m), 7.47-7.43 (3H, m), 7.35 (1H, t, J 7.4 Hz), 7.17 (1H, t, J 7.5 Hz), 3.89 (3H, s), 3.59 (4H, s), 2.33 (3H, s), 1.90 (4H, s); MS (ES⁺) m/z 405 [M+H]⁺.

### EXAMPLE 38

### 3-[2-(Dim ethylamino)ethoxy]-2-phenyl-4-quinolinecarboxylic acid, 2-(methoxycarbonyl)-2-phenylhydrazide

To a solution of 3-hydroxy-2-phenyl-4-quinolinecarboxylic acid, 2-(methoxycarbonyl)-2-phenylhydrazide (Description 6, 0.2 g, 0.48 mmol) in THF (10 mL) was added K₂CO₃ (0.2 g, 1.44 mmol), 2-chloro-N,N-dimethylethanamine hydrochloride (0.076 g, 0.53 mmol) and sodium iodide (5 mg). The mixture was heated at 60° C. for 16 h., cooled, filtered and the solvent was evaporated under reduced pressure. The residue was purified by chromatography on silica gel using 0-4% methanol in DCM as eluent to give the title compound as a foam (34 mg).

¹H NMR (500 MHz, CDCl₃): 12.80 (1H, br, s), 8.10 (2H, t), 7.94 (2H, d), 7.67-7.40 (9H, m), 7.24 (1H, t), 3.92 (3H, s), 3.76 (2H, br s), 2.26 (2H, br, s), 1.91 (6H, br, s). m/z (ES⁺) 485 [M+H⁺].

### EXAMPLE 39

### 3-[2-(4-Morpholinyl)ethoxy]-2-phenyl-4-quinolinecarboxylic acid, 2-(methoxycarbonyl)-2-phenylhydrazide

To a solution of methyl 3-hydroxy-2-phenyl-4-quinolinecarboxylic acid, 2-(methoxycarbonyl)-2-phenylhydrazide (Description 6, 0.2 g, 0.48 mmol) in THF (10 mL) was added K₂CO₃ (0.2 g, 1.44 mmol), N-(2-chloroethyl)morpholine hydrochloride (0.098 g, 0.53 mmol) and sodium iodide (5 mg). The mixture was heated at 60° C. for 48 h., cooled, filtered and the solvent was evaporated under reduced pressure. The residue was purified by chromatography on silica gel using 0-100% EtOAc in DCM as eluent to give the title compound as a foam (56 mg).

¹H NMR (500 MHz, CDCl₃): 11.71 (1H, br, s), 8.12 (2H, t), 7.93 (2H, d), 7.70-7.40 (9H, m), 7.24 (1H, t), 3.94 (3H, s), 3.79 (2H, br s), 3.43 (4H, s), 2.30 (2H, br, s), 2.07 (4H, s). m/z (ES⁺) 527 [M+H⁺].

### EXAMPLE 40

### 3-(4-Morpholinylmethyl)-2-phenyl-4-quinolinecarboxylic acid, 2-(methoxycarbonyl)-2-phenylhydrazide

To a solution of 3-(bromomethyl)-2-phenyl-4-quinolinecarboxylic acid, 2-(methoxycarbonyl)-2-phenylhydrazide (0.146 g, Description 10) in THF (3 mL) was added a solution of morpholine (0.047 mL) in THF (3 mL) and the mixture was stirred at rt for 60 min. The solvent was evaporated under reduced pressure and the residue was purified by chromatography on silica gel (eluting with 0% to 40% EtOAc in isohexane). The residue was dissolved in ether and ethereal hydrogen chloride (1M, 0.3 mL) was added. The solvent was evaporated under reduced pressure and the residue was washed with isohexane and dried in vacuo to give the title compound.

¹H NMR (360 MHz, DMSO d6,) δ 11.8 (1H, broad s), 8.10 (1H, d J 8.4 Hz), 8.01 (1H, broad s), 7.88 (1H, t J 7.7 Hz), 7.71 (1H, t J 7.6 Hz), 7.56-7.43 (7H, m), 7.35 (1H, t J 7.1 Hz), 4.1-3.1 (13H, broad m). m/z (ES⁺) 497 [M+H⁺].

### EXAMPLE 41

### 3-[(Cyclohexylmethylamino)methyl]-2-phenyl-4-quinolinecarboxylic acid, 2-(methoxycarbonyl)-2-phenylhydrazide

To a solution of 3-(chloromethyl)-2-phenyl-4-quinolinecarboxylic acid, 2-(methoxycarbonyl)-2-phenylhydrazide (Description 12) (30 mg, 0.0672 mmol) in THF (2 mL) was added N-methylcyclohexylamine (17.6 μL, 0.135 mmol) and the mixture was heated under reflux overnight. The mixture was cooled, the solvent was evaporated under reduced pressure and the residue was purified by LC-MS to give the title compound (24.5 mg, 70%). ¹H NMR (400 MHz, DMSO-d₆) δ 8.04 (1H, d, J 8.3 Hz), 7.82 (1H, J 7.7 Hz), 7.69 (1H, broad t), 7.53-7.38 (10H, m), 7.31 (1H, t, J=7.3 Hz), 3.81 (3H, s), 1.73 (1H, m), 1.61 (3H, s), 1.47 (2H, m), 1.41 (1H, m), 1.02 (2H, m), 0.91-0.69 (5H, m).

1-37. (canceled)

38. A compound of the formula (I): 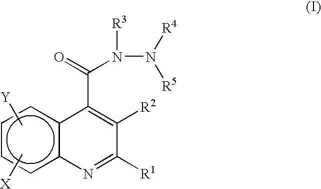
wherein:
R¹ is a phenyl, naphthyl or heteroaryl ring, wherein heteroaryl is a 5-membered unsaturated ring containing 1, 2, 3 or 4 nitrogen atoms and/or, an oxygen or sulphur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;
or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄ alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a}, where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen, or, when R¹ is CONR^{a}R^{b} or SO₂NR^{a}R^{b}, R^{a}, R^{b}, and the nitrogen atom to which they are attached form a piperidine, piperazine, pyrrolidine, morpholine, aziridine, azetidine or azepine ring, optionally substituted by hydroxy, C₁₋₄alkyl or C₁₋₄alkoxy;
R² is hydrogen, hydroxy, halogen or CN;
or R² is C₁₋₆alkyl, C₂₋₆alkenyl, C₂₋₆alkynyl, C₃₋₈cycloalkyl, C₁₋₆alkoxy, (CH₂)₀₋₆NR^{c}R^{d}, C₁₋₆alkoxy substituted by NR^{c}R^{d}, OC₃₋₈cycloalkyl, OHet, Het, Oheteroaryl, heteroaryl, Oaryl, aryl, (CH₂)₀₋₄NR^{e}C(O)R^{f}, (CH₂)₀₋₄NR^{e}C(O)OR^{f}, (CH₂)₀₋₄NR^{e}S(O)₂R^{f}, SO₂R^{c}, SO₂NR^{c}R^{d}, COOR^{c}, C(O)R^{c}, C(O)NR^{c}R^{d},
optionally substituted by 1 to 8 halogen atoms,
where R^{c}, R^{d}, R^{e} and R^{f} are independently selected from hydrogen, C₁₋₆alkyl, C₂₋₆alkenyl, C₂₋₆alkynyl, C₃₋₈cycloalkyl and aryl,
or where R^{c} and R^{d}, together with the nitrogen atom to which they are attached, form a saturated nitrogen-containing 3-7 membered heterocycle optionally containing a further nitrogen or oxygen atom and optionally substituted by NR′R″, where R′ and R″ are independently chosen from hydrogen and C₁₋₆alkyl, or R^{e} and R^{f} are linked as a C₂₋₆alkylene, C₂₋₆alkenylene or C₃₋₆alkynylene group, optionally substituted by hydroxyl or halogen,
where Het is as hereinbelow defined;
R³ is hydrogen or C₁₋₆alkyl;
R⁴ is hydrogen, C₁₋₈alkyl, C₂₋₈alkenyl, C₂₋₈alkynyl, C₃₋₈cycloalkyl, aryl or arylC₁₋₆alkyl, optionally substituted by hydroxy, C₁₋₆alkoxy, CN, NH₂ or 1 to 8 halogen atoms;
or R⁴ is a moiety containing at least one aromatic ring and possessing 5, 6, 9 or 10 ring atoms of which 1, 2, 3 or 4 atoms are heteroatoms independently selected from N, O and S, which ring system is optionally substituted at any substitutable position by 1, 2 or 3 groups chosen from hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, which group is optionally substituted by 1 to 8 halogen atoms;
R⁵ is hydrogen, C₁₋₈alkyl, C₂₋₈alkenyl, C₂₋₈alkynyl, C₃₋₈cycloalkyl, aryl, arylC₁₋₆ alkyl, (CH₂)₀₋₄heteroaryl, (CH₂)₀₋₄Het, C(O)NR^{g}R^{h}, S(O)₂NR^{g}R^{h}, S(O)₂R^{g}, C(O)OR^{g} or C(O)R^{g},
where R^{g} and R^{h} are each independently selected from hydrogen, C₁₋₆alkyl, C₂₋₆alkenyl, C₂₋₆alkynyl, (CH₂)₀₋₄C₃₋₈-cycloalkyl, (CH₂)₀₋₄aryl, (CH₂)₀₋₄heteroaryl and (CH₂)₀₋₄Het, optionally substituted by hydroxy and 1 to 8 halogen atoms;
or R⁴ and R⁵, together with the nitrogen atom to which they are attached, form a mono- or bicyclic moiety possessing 3 to 10 ring atoms of which optionally 1, 2, 3 or 4 atoms are heteroatoms independently selected from N, O and S, which ring system is optionally substituted at any substitutable position by 1, 2 or 3 groups chosen from halogen, NO₂, CN, NH₂, oxo, C₁₋₄alkyl, C₂₋₄alkenyl, C₂₋₄alkynyl and C₁₋₆alkoxy, which group is optionally substituted by 1 to 8 halogen atoms;
X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NRⁱRⱼ, CO₂Rⁱ, (CH₂)₀₋₄NR^{k}R^{m}, SO₂Rⁱ and SO₂NRⁱR^{j}, optionally substituted by halogen;
Rⁱ and Rⱼ are independently chosen from hydrogen, C₁₋₆alkyl, C₂₋₆alkenyl, C₂₋₆alkynyl, C₃₋₈cycloalkyl and (CH₂)₀₋₄aryl;
R^{k} and R^{m} are independently chosen from hydrogen, C₁₋₆alkyl, C₂₋₆alkenyl, C₂₋₆alkynyl, C₃₋₈-cycloalkyl, aryl, C(O)R^{p} and S(O)₂R^{p};
R^{p} is hydrogen, C₁₋₆alkyl, C₂₋₆alkenyl, C₂₋₆alkynyl, C₃₋₈cycloalkyl or (CH₂)₀₋₄aryl;
or a pharmaceutically acceptable salt thereof;
with the proviso that the compound of the formula (I) is not: 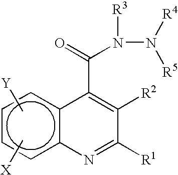
wherein:
R¹ is a phenyl, naphthyl or heteroaryl ring, wherein heteroaryl is a 5-membered unsaturated ring containing 1, 2, 3 or 4 nitrogen atoms and/or, an oxygen or sulphur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;
R² is hydroxy, C₁₋₆alkoxy, C₁₋₆alkyl, amino, NR′R″ or C₁₋₆alkyl-NR′R″ where R′ and R″ are independently chosen from hydrogen and C₁₋₄alkyl and where R′ and R″, together with the nitrogen atom to which they are attached, form a saturated nitrogen-containing 3-7 membered heterocycle optionally containing a further nitrogen atom and optionally substituted by NR′R″ as defined above or R² is C₁₋₆alkoxy substituted by NR′R″ as defined above;
R³ is hydrogen or C₁₋₆alkyl;
R⁴ is hydrogen, C₁₋₈alkyl, C₂₋₈alkenyl, C₂₋₈alkynyl, C₃₋₈cycloalkyl, aryl or arylC₁₋₆alkyl;
R⁵ is hydrogen, C₁₋₈alkyl, C₂₋₈alkenyl, C₂₋₈alkynyl, C₃₋₈cycloalkyl, aryl, arylC₁₋₆alkyl or C₁₋₆alkoxycarbonyl;
or R⁴ and R⁵, together with the nitrogen atom to which they are attached, form a C₃-C₁₀ mono- or bicyclic saturated ring;
X and Y are independently chosen from hydrogen, hydroxy, nitro, amino, cyano, CF₃, halogen and C₁₋₄alkyl;
or a pharmaceutically acceptable salt thereof.

39. The compound of claim 38 where R¹ is a phenyl, naphthyl or heteroaryl ring, optionally substituted by 1, 2 or 3 groups independently chosen from hydroxy, halogen, NO₂, CN, NH₂, CF₃, C₁₋₄alkyl, C₂₋₄alkenyl and C₂₋₄alkynyl.

40. The compound of claim 39 where R¹ is 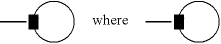
is defined as a 4- to 7-membered heteroaliphatic ring, containing 1, 2 or 3 heteroatoms selected from N, O or S or a group S(O), S(O)₂, NH or NC₁₋₄alkyl, which ring is attached through the nitrogen atom, optionally substituted by 1, 2 or 3 groups independently chosen from hydroxy, halogen, NO₂, CN, NH₂, CF₃, C₁₋₄alkyl, C₂₋₄alkenyl and C₂₋₄alkynyl.

41. The compound of claim 40 where R¹ is aziridine, azetidinyl, pyrrolidinyl, piperidinyl, azepinyl or piperazinyl, optionally substituted by 1, 2 or 3 groups independently chosen from hydroxy, halogen, NO₂, CN, NH₂, CF₃, C₁₋₄alkyl, C₂₋₄alkenyl and C₂₋₄alkynyl.

42. The compound of claim 38 where R² is C₁₋₆alkyl, C₁₋₆alkoxy or NR^{c}R^{d}, substituted by 1 to 8 halogen atoms.

43. The compound of claim 42 where R² is CH₂F, CHF₂, CF₃, CH₂CF₃, OCF₃, OCH₂CF₃, N(H)CF₃, N(H)CH₂CF₃, N(CH₃)CF₃, N(CH₃)CH₂CF₃, N(CF₃)₂, N(CF₃)CH₂CF₃, N(CH₂CF₃)₂ or N(CH₂CH₃)CH₂CF₃.

44. The compound of claim 43 where R² is N(H)CH₂CF₃ or OCH₂CF₃.

45. The compound of claim 38 where R⁴ is phenyl or a five- or six-membered aromatic ring containing 1, 2 or 3 heteroatoms selected from N, O and S, optionally substituted by 1, 2 or 3 groups chosen from halogen, CF₃, C₁₋₄alkyl and CN.

46. The compound of claim 38 where R⁵ is hydrogen, C₁₋₆alkyl, aryl, S(O)₂R^{g}, C(O)OR^{g} or C(O)R^{g}, where R^{g} is as defined in claim 38.

47. The compound of claim 46 where R⁵ is hydrogen, C₁₋₄alkyl, phenyl, S(O)₂R^{g} or C(O)R^{g}, where R^{g} is C₁₋₆alkyl or heteroaryl.

48. The compound of claim 47 where R⁵ is hydrogen, methoxycarbonyl, ethyl, methyl, phenyl, S(O)₂CH₃, C(O)CH₃,

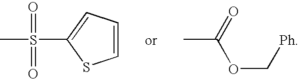

49. The compound of claim 38 where X and Y are independently hydrogen or methyl.

50. The compound of claim 49 where X and Y are both hydrogen.

51. The compound of claim 38 of the formula (Ia): 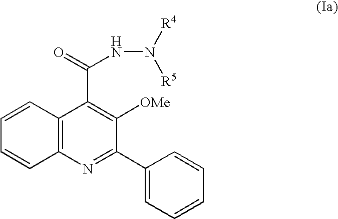
wherein:
R⁵ is C(O)NR^{g}R^{h}, S(O)₂NR^{g}R^{h}, S(O)₂R^{g}, C(O)OPh, C(O)OCH₂Ph or C(O)R^{g},
or a pharmaceutically acceptable salt thereof.

52. The compound of claim 51 where R⁴ is phenyl, pyridyl, pyrimidinyl or benzothiazolyl, optionally substituted by 1 or 2 groups chosen from halogen, CF₃, methyl, ethyl and CN.

53. The compound of claim 51 where R⁵ is S(O)₂R^{g}, C(O)OCH₂Ph or C(O)R^{g}, where R^{g} is selected from methyl, ethyl, iso-propyl, benzyl, (CH₂)₀₋₁heteroaryl, phenyl, (CH₂)₀₋₁C₃₋₈cycloalkyl and (CH₂)₀₋₁Het, optionally substituted by 1 to 5 fluorine atoms.

54. The compound of claim 38 of formula (Ib): 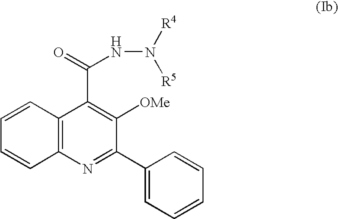
wherein:
R⁴ is C₁₋₈alkyl, C₂₋₈alkenyl, C₂₋₈alkynyl, C₃₋₈cycloalkyl, aryl or arylC₁₋₆alkyl, substituted by hydroxyl or 1 to 8 halogen atoms,
or R⁴ is a moiety containing at least one aromatic ring and possesses 5, 6, 9 or 10 ring atoms of which 1, 2, 3 or 4 atoms are heteroatoms independently selected from N, O and S, which ring system is optionally substituted at any substitutable position by 1, 2 or 3 groups chosen from hydroxy, halogen, NO₂, CN, NH₂, C₁₋₄alkyl, C₂₋₄alkenyl, C₂₋₄alkynyl, C₁₋₄alkoxy and C(O)OC₁₋₆alkyl, which group is optionally substituted by 1 to 8 halogen atoms,
or a pharmaceutically acceptable salt thereof.

55. The compound of claim 54 where R⁴ is phenyl substituted by 1 or 2 groups chosen from halogen, CF₃, OCF₃, methyl, ethyl and CN, or R⁴ is pyridyl, pyrimidinyl or benzothiazolyl, optionally substituted by 1 or 2 groups chosen from halogen, C₁₋₄alkyl, C₁₋₄alkoxy, CF₃, OCF₃ and NH₂.

56. The compound of claim 54 where R⁵ is hydrogen, C₁₋₆ alkyl, aryl, S(O)₂R^{g}, C(O)OR^{g} or C(O)R^{g}.

57. The compound of claim 56 where R⁵ is S(O)₂R^{g}, C(O)OCH₂P^{h} or C(O)R^{g}, where R^{g} is selected from methyl, ethyl, iso-propyl, (CH₂)₀₋₁phenyl, (CH₂)₀₋₁C₃₋₈cycloalkyl, (CH₂)₀₋₁heteroaryl and (CH₂)₀₋₁Het, optionally substituted by 1 to 5 fluorine atoms.

58. The compound of claim 38 of the formula (Ic): 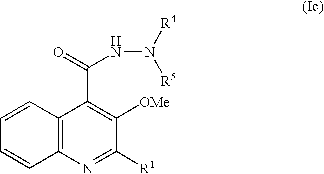
wherein:
R¹ is a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a},
or a pharmaceutically acceptable salt thereof.

59. The compound of claim 58 where R¹ is Het.

60. The compound of claim 59 where R¹ is

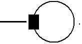

61. The compound of claim 60 where R¹ is pyrrolidinyl, piperidinyl or piperazinyl.

62. The compound of claim 58 where R⁴ is phenyl, pyridyl, pyrimidinyl or benzothiazolyl, optionally substituted by 1 or 2 groups chosen from halogen, CF₃, methyl, ethyl and CN.

63. The compound of claim 58 where R⁵ is hydrogen, C₁₋₆ alkyl, aryl, S(O)²R^{g}, C(O)OR^{g} or C(O)R^{g}, where R^{g} is as defined in claim 38.

64. The compound of claim 63 where R⁵ is S(O)₂R^{g}, C(O)OCH₂Ph or C(O)R^{g}, where R^{g} is selected from methyl, ethyl, iso-propyl, (CH₂)₀₋₁phenyl, (CH₂)₀₋₁C₃₋₈cycloalkyl, (CH₂)₀₋₁heteroaryl and (CH₂)₀₋₁Het, optionally substituted by 1 to 5 fluorine atoms.

65. The compound of claim 38 of the formula (Id): 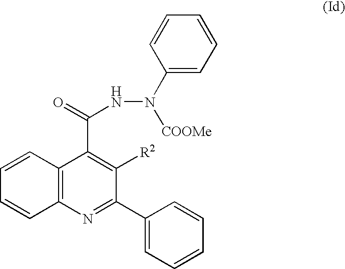
or a pharmaceutically acceptable salt thereof.

66. The compound of claim 65 where R² is (CH₂)₀₋₆NR^{c}R^{d} or C₁₋₆alkoxy substituted by NR^{c}R^{d}.

67. The compound of claim 66 where R² is CH₂NR^{c}R^{d} or OCH₂CH₂NR^{c}R^{d}.

68. A compound which is selected from the group consisting of:
methyl 1-(2-fluorophenyl)-2-[(3-methoxy-2-phenylquinolin-4-yl)carbonyl]hydrazinecarboxylate,
methyl 1-(3-fluorophenyl)-2-[(3-methoxy-2-phenylquinolin-4-yl)carbonyl]hydrazinecarboxylate,
methyl 1-(4-fluorophenyl)-2-[(3-methoxy-2-phenylquinolin-4-yl)carbonyl]hydrazinecarboxylate,
methyl 1-(2-chlorophenyl)-2-[(3-methoxy-2-phenylquinolin-4-yl)carbonyl]hydrazinecarboxylate,
methyl 1-(3-chlorophenyl)-2-[(3-methoxy-2-phenylquinolin-4-yl)carbonyl]hydrazinecarboxylate,
methyl 1-(4-chlorophenyl)-2-[(3-methoxy-2-phenylquinolin-4-yl)carbonyl]hydrazinecarboxylate,
methyl 2-[(3-methoxy-2-phenylquinolin-4-yl)carbonyl]-1-(2-methylphenyl)hydrazinecarboxylate,
methyl 2-[(3-methoxy-2-phenylquinolin-4-yl)carbonyl]-1-(3-methylphenyl)hydrazinecarboxylate,
methyl 2-[(3-methoxy-2-phenylquinolin-4-yl)carbonyl]-1-(4-methylphenyl)hydrazinecarboxylate,
methyl 1-(2-ethylphenyl)-2-[(3-methoxy-2-phenylquinolin-4-yl)carbonyl]hydrazinecarboxylate,
methyl 2-[(3-methoxy-2-phenylquinolin-4-yl)carbonyl]-1-[3-(trifluoromethyl)phenyl]-hydrazinecarboxylate,
methyl 1-(4-bromophenyl)-2-[(3-methoxy-2-phenylquinolin-4-yl)carbonyl]hydrazinecarboxylate,
methyl 1-(4-iodophenyl)-2-[(3-methoxy-2-phenylquinolin-4-yl)carbonyl]hydrazinecarboxylate,
methyl 1-(4-cyanophenyl)-2-[(3-methoxy-2-phenylquinolin-4-yl)carbonyl]hydrazinecarboxylate,
methyl 2-[(3-methoxy-2-phenylquinolin-4-yl)carbonyl]-1-[4-(trifluoromethoxy)phenyl]-hydrazinecarboxylate,
methyl 1-(2,5-difluorophenyl)-2-[(3-methoxy-2-phenylquinolin-4-yl)carbonyl]hydrazinecarboxylate,
methyl 1-(3-chloro-4-fluorophenyl)-2-[(3-methoxy-2-phenylquinolin-4-yl)carbonyl]-hydrazinecarboxylate,
methyl 1-(2,6-dichlorophenyl)-2-[(3-methoxy-2-phenylquinolin-4-yl)carbonyl]hydrazinecarboxylate,
methyl 1-(3,5-dichlorophenyl)-2-[(3-methoxy-2-phenylquinolin-4-yl)carbonyl]hydrazinecarboxylate,
methyl 1-(2,5-dichlorophenyl)-2-[(3-methoxy-2-phenylquinolin-4-yl)carbonyl]hydrazinecarboxylate,
methyl 1-(3-chloro-4-methylphenyl)-2-[(3-methoxy-2-phenylquinolin-4-yl)carbonyl]-hydrazinecarboxylate,
methyl 1-[2-chloro-5-(trifluoromethyl)phenyl]-2-[(3-methoxy-2-phenylquinolin-4-yl)carbonyl]hydrazinecarboxylate,
methyl 2-[(3-methoxy-2-phenylquinolin-4-yl)carbonyl]-1-pyridin-2-ylhydrazinecarboxylate,
methyl 2-[(3-methoxy-2-phenylquinolin-4-yl)carbonyl]-1-[4-(trifluoromethyl)pyrimidin-2-yl]hydrazinecarboxylate,
methyl 1-(1,3-benzothiazol-2-yl)-2-[(3-methoxy-2-phenylquinolin-4-yl)carbonyl]hydrazinecarboxylate,
benzyl 2-[(3-methoxy-2-phenylquinolin-4-yl)carbonyl]-1-phenylhydrazinecarboxylate,
N′-acetyl-3-methoxy-N′,2-diphenylquinoline-4-carbohydrazide,
N′-isobutyryl-3-methoxy-N′,2-diphenylquinoline-4-carbohydrazide,
N′-(cyclopentylacetyl)-3-methoxy-N′,2-diphenylquinoline-4-carbohydrazide,
N′-(4-fluorobenzoyl)-3-methoxy-N′,2-diphenylquinoline-4-carbohydrazide,
N′-2-furoyl-3-methoxy-N′,2-diphenylquinoline-4-carbohydrazide,
3-methoxy-N′,2-diphenyl-N′-(phenylacetyl)quinoline-4-carbohydrazide,
3-methoxy-N′,2-diphenyl-N′-(2-thienylacetyl)quinoline-4-carbohydrazide,
3-methoxy-N′-(methylsulfonyl)-N′,2-diphenylquinoline-4-carbohydrazide,
3-methoxy-N′,2-diphenyl-N′-(2-thienylsulfonyl)quinoline-4-carbohydrazide,
3-methoxy-N′-(morpholin-4-ylcarbonyl)-N′,2-diphenylquinoline-4-carbohydrazide, methyl 2-[(3-methyl-2-pyrrolidin-1-ylquinolin-4-yl)carbonyl]-1-phenylhydrazine-carboxylate,
3-[2-(dimethylamino)ethoxy]-2-phenyl-4-quinolinecarboxylic acid, 2-(methoxycarbonyl)-2-phenylhydrazide,
3-[2-(4-morpholinyl)ethoxy]-2-phenyl-4-quinolinecarboxylic acid, 2-(methoxycarbonyl)-2-phenylhydrazide,
3-(4-morpholinylmethyl)-2-phenyl-4-quinolinecarboxylic acid, 2-(methoxycarbonyl)-2-phenylhydrazide,
3-[(cyclohexylmethylamino)methyl]-2-phenyl-4-quinolinecarboxylic acid, 2-(methoxycarbonyl)-2-phenylhydrazide,
or a pharmaceutically acceptable salt thereof.

69. A pharmaceutical composition comprising the compound of claim 38 or a pharmaceutically acceptable salt thereof and a pharmaceutically acceptable excipient.

70. A method of treatment of a patient suffering from a neurokinin-2 and/or neurokinin-3 mediated disease, which comprises administering to the patient a therapeutically effective amount of the compound of claim 38 or a pharmaceutically acceptable salt thereof.

71. A method of treatment of schizophrenia in a patient in need thereof which comprises administering to the patient a therapeutically effective amount of the compound of claim 38 or a pharmaceutically acceptable salt thereof.
